(19) United States
(12) Patent Application Publication
(10) Pub. No.: US 2025/0332364 A1
(43) Pub. Date: Oct. 30, 2025

(54) Hand-Held Device for Personalized Mechanical Ventilation Settings Using Structured Light Near-Infrared Imaging

(71) Applicant: Black Fur Biomedical, LLC, Tucson, AZ (US)

(72) Inventors: Ryan REDFORD, Irvine, CA (US); Elizabeth ROHRS, Tucson, AZ (US); Steven REYNOLDS, Tucson, AZ (US)

(21) Appl. No.: 19/195,314

(22) Filed: Apr. 30, 2025

Related U.S. Application Data
(60) Provisional application No. 63/640,775, filed on Apr. 30, 2024.

(51) Int. Cl. A61M 16/00

(52) U.S. Cl. CPC A61M 16/024; A61M 2205/3313; A61M 2205/3327

(57) ABSTRACT

A medical device and method for providing personalized ventilation settings using a hand-held tool that employs structured light near-IR imaging to accurately map patient torso dimensions. The device integrates advanced optical components and AI-driven analysis to offer safer, more precise, and individualized mechanical ventilation strategies, particularly enhancing care for female patients at risk of over-ventilation.

### CROSS REFERENCE TO RELATED APPLICATION

This application claims priority from U.S. Provisional Application Ser. No. 63/640,775, filed Apr. 30, 2024, the contents of which are incorporated herein in their entirety.

### FIELD OF THE INVENTION

The present invention relates generally to medical devices and more particularly to a system and method for measuring and analyzing body dimensions to provide personalized mechanical ventilation settings. This disclosure has particularly utility for determining proper settings and/or ventilation settings dependent on patient data and calculations performed therewith.

### BACKGROUND OF THE INVENTION

This section provides background information related to the present disclosure which is not necessarily prior art. This section provides a general summary of the disclosure and is not a comprehensive disclosure of its full scope or all its features.

Mechanical ventilation is a critical intervention in critical care settings but comes with risks, particularly ventilator-induced lung injuries. Current methods estimate lung volume based primarily on height, leading to suboptimal ventilation settings, especially for female patients, who often suffer from over-ventilation due to inaccurate estimations of their body dimensions. The instant disclosure assists in obtaining personalized ventilation settings using non-invasive techniques.

### SUMMARY OF THE INVENTION

The invention provides a hand-held, non-invasive imaging device designed to improve the accuracy of lung volume estimations and mechanical ventilation settings. This device utilizes structured light near-infrared (IR) technology to scan and create detailed 3D maps of a patient's torso. By precisely measuring factors such as height, torso girth, and volume, the device can calculate personalized ventilation settings that are tailored to the patient's specific physiology, significantly reducing the risk of ventilator-induced injuries.

Embodiments of the present disclosure provide an imaging device and system for calculating ventilation settings. Briefly described, one embodiment of the system, among others, can be implemented as follows. The instant disclosure provides a method for determining personalized ventilation settings for a patient in need of ventilation support, comprising the steps of using an emitting structured light to capture 3D spatial data of a patient's torso; analyzing the data with a program to compute critical measurements including torso height, girth, and volume; and utilizing these measurements to recommend personalized mechanical ventilation settings.

In one aspect, the device comprises a hand-held device.

In another aspect, the structured light is in the near-infrared spectrum, approximately around 940 nm, to maximize penetration and minimize interference from ambient light.

In yet another aspect, the computational analysis program processes the data to calculate personalized settings, specifically tailoring ventilation parameters based on the unique anatomical data of the patient.

In yet another aspect, the system comprises a device configured to emit structured light to capture 3D special data of a patient's torso and other anatomical features and provides for a computer configured to store and analyze the data using a program to algorithmically compute critical measurements including torso height, girth and volume.

In another aspect, the system device is a hand-held device comprising structured light which is near-infrared technology within the spectrum of approximately 940 nm.

In yet another aspect, the computational analysis program processes the data within an output device utilizing a processor to calculate personalized settings and store said computations in non-transitory memory, said computations specifically tailoring ventilation parameters based on the unique anatomical data of the patient.

In another aspect, the handheld device is employed for measuring a patient's attributes prior to intubation, wherein the device obtains measurements and comprises a processor and non-transitory memory.

In yet another aspect, the handheld device utilizes structured light near-infrared (IR) technology scans and creates 3D maps of a patient's body, wherein the device provides data that can be utilized to calculate ventilation settings based on the patient's physiology.

In another aspect, the data from the handheld device can be shared with output devices, i.e., tablets, smartphones, computers or the like.

In another aspect, the handheld device measures physiological attributes such as torso girth, height and volume.

### BRIEF DESCRIPTION OF THE DRAWINGS

Further features and advantages of the disclosure will be seen in the following detailed description, taken in conjunction with the accompanying drawings. The drawings described herein are for illustrative purposes only of selected embodiments and not all possible implementations and are not intended to limit the scope of the present disclosure.

In the drawings:

FIG. 1 depicts a clinician uses handheld device for ventilation settings in accordance with the present invention.

FIG. 2 depicts a handheld device in accordance with the present invention.

FIG. 3 depicts a flowchart for noise reduction filters.

### DETAILED DESCRIPTION OF THE INVENTION

Example embodiments will now be described more fully with reference to the accompanying drawings. Example embodiments are provided so that this disclosure will be thorough and will fully convey the scope to those who are skilled in the art. Numerous specific details are set forth such as examples of specific components, devices, and methods to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail.

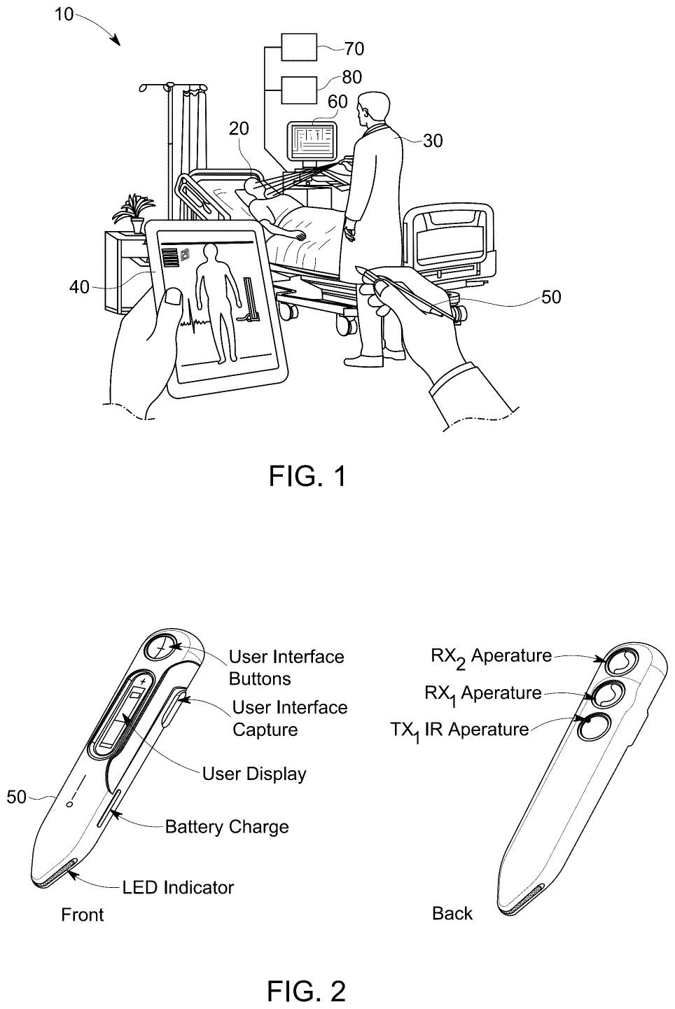

FIG. 1 depicts a clinician using a handheld device for ventilation settings in accordance with the present invention. As shown, within system 10, patient 20 is scanned by handheld device 50 by physician or healthcare professional/clinician 30. The patient's attributes are recorded within a non-transitory memory 80 within said device 50 and can be parsed to an output device, i.e., a tablet 40, computer 60 or the like wherein a processor 70 may perform important calculations prior to intubation of patient 20. The patient data can be stored within healthcare providers databases to eliminate the need to re-calculate for future procedures. Other input devices, i.e., cameras, scanners, SD cards may be utilized to provide more data to the system prior to calculation. Likewise, many other output devices may be used to display the results, i.e., monitors, tablets, phones, and the like.

FIG. 2 depicts a handheld device in accordance with the present invention. As can be seen, the device 50 provides interface buttons for navigation and capturing of patient 20 measurements. Device 50 has a display to view the associated measurements as obtained by the aperture lenses (RX1 and RX2). An LED and battery charge indicator lights are provided for the status of device 50.

The connection between patient height, torso mass, and ventilation settings is critical in mechanical ventilation, which is a life-saving intervention for patients who are unable to breathe adequately on their own. These measurements are important for several reasons, as will be described below.

The first attribute that one must take into account is patient height. Patent height relates to the following:

Lung Size Correlation: A person's height is closely related to the size of their lungs. Taller individuals typically have larger lungs with greater volumes. Ventilation settings, particularly tidal volume (the volume of air delivered to the lungs with each breath from the ventilator), are often calculated based on predicted lung volume, which is estimated from height.

Avoiding Overdistention: Without accurate height measurements, the risk is setting a tidal volume that is too high, which can lead to overdistention of the lungs. This can damage the delicate alveoli where gas exchange occurs.

Preventing Atelectasis: Conversely, setting a tidal volume that is too low can lead to atelectasis, which is the collapse of lung tissue, affecting oxygenation and potentially leading to infection.

Another attribute that is important when intubating a patient is torso mass. This relates to the following:

Thoracic Pressure Influence: The mass of the torso, which includes muscle, bone, and adipose tissue, can influence the pressure within the thoracic cavity. This pressure impacts how easily the lungs can expand and contract.

Ventilator Induced Lung Injury (VILI): Excessive pressure or volume delivered to the lungs can cause VILI. A correct estimation of torso mass helps adjust the ventilator pressure to avoid such injuries.

Personalized Care: Women, in particular, can have variable distributions of torso mass due to differences in breast tissue, which can affect lung compliance (how easily the lung can expand). Children, in particular infants and adolescents, also can have variable distributions of torso mass. Personalized settings based on accurate torso mass measurements can accommodate these differences.

Pregnancy Considerations: For pregnant women, the growing fetus increases the intra-abdominal pressure, which can affect lung mechanics. Accurate torso mass measurements help adjust ventilation strategies to accommodate these changes.

In summary, height and torso mass provide essential data for calculating the size and compliance of the lungs, which are necessary to tailor mechanical ventilation settings to the individual patient's needs. This personalization aims to optimize oxygen and carbon dioxide exchange while minimizing potential harm, improving outcomes for patients who require ventilatory support.

The instant disclosure provides a structured light near-infrared (IR) imaging system to assist in obtaining proper measurements. The Structured Light Near-Infrared (IR) Imaging System provides components, wherein the device consists of dual apertures and high-resolution CMOS sensors that project a structured grid pattern of near-IR light onto the patient's body. The light pattern is altered by the contours of the body, allowing the device to capture detailed spatial data.

Further, the Structured Light Near-Infrared (IR) Imaging System provides operative features, wherein the device processes these alterations to construct a 3D map that accurately reflects body dimensions crucial for calculating lung volume and ventilation needs.

Additionally, the Structured Light Near-Infrared (IR) Imaging System provides an AI-Powered Computational Analysis Program, wherein the integrated software uses novel machine learning algorithms to analyze the captured 3D data. It assesses height, torso girth, and volume distribution to determine the optimal mechanical ventilation settings.

The benefits produced by utilizing the foregoing system is to reduce human error in manually estimating physical dimensions, providing a more accurate, reproducible, and safer approach to ventilator settings.

Mathematics and algorithms primarily involve geometrical optics, computational algorithms for 3D reconstruction, and predictive modeling based on anatomical measurements. Provided herewith are foundational equations and their descriptions that can be used.

### 1. Geometrical Reconstruction of 3D Surfaces

The structured light imaging technique involves projecting a known pattern onto a three-dimensional surface and capturing the deformation of this pattern due to the surface's shape. The basic principle can be described with the triangulation method:

$$z = \frac{b \cdot f}{d}$$

wherein
z is the distance from the camera to the point on the object's surface.
b is the baseline, or the distance between the structured light source and the camera.
f is the focal length of the camera.
d is the displacement of the light pattern observed by the camera from its original position.

This equation helps calculate the depth information (z) required to reconstruct the 3D surface of the patient's torso.

2. Volume Calculation from Surface Data

Once the 3D surface is reconstructed, the next step is calculating the volume, which is critical for determining lung capacity. The volume V of the torso can be approximated by integrating the surface area over the depth obtained from the 3D map:

$$v = {\int }_{A} zdA$$

wherein
A represents the projected area of the torso on the plane perpendicular to the beam.
z is the depth at each point, integrated over the entire area.

### 3. Predictive Modeling for Ventilation Settings

To predict personalized ventilation settings based on the reconstructed torso volume, regression analysis or machine learning models can be used. An example model could be a linear regression model where tidal volume Vₜ is predicted based on body measurements such as torso volume V:

$${V}_{t} = α + β V$$

α and β are model coefficients determined from training data (clinical trial data, for example).
V is the calculated torso volume from the 3D imaging system.

### 4. Safety Margin and Compliance Calculation

The device can also be programmed to calculate the safe range of pressures to avoid overventilation. The compliance c of the lung can be estimated by:

$$c = \frac{Δ V}{Δ p}$$

ΔV is the change in lung volume.
Δp is the change in transpulmonary pressure.

This compliance can be used to adjust the pressure settings on the ventilator to ensure that it is within safe limits, thus preventing ventilator-induced lung injuries.

When dealing with geometric reconstruction, particularly when considering noise correction (which could stem from various sources, including patient movement, ambient light interference, or sensor inaccuracies), several mathematical approaches and equations can be used to refine the data and enhance the accuracy of the reconstruction. The foregoing approaches serve this purpose:

### 1. Least Squares Method

This is used to minimize the error (noise) in measurements and data points. The least squares fitting can be expressed by:

$$\hat{x} = {( {A}^{T} A )}^{- 1} {A}^{T} b$$

wherein:
A is the matrix of observed values,
b is the vector of measurements,
\hat{x} is the vector of the estimated parameters.

### 2. Kalman Filter

The Kalman filter is a powerful mathematical algorithm used for estimating the state of a system based on noisy measurements. While it finds applications in various fields, including robotics, economics, and control systems, the focus is on its role in 3D image capturing.

3D Reconstruction and Structure from Motion (SfM)

In 3D reconstruction tasks, the Kalman filter assists in estimating 3D points and camera positions across frames.

When capturing images from different viewpoints (e.g., using multiple cameras), the Kalman filter helps refine the 3D structure by combining predictions and measurements.

It handles noisy or conflicting measurements to provide a more accurate estimate of the 3D scene.

### Optimized Design of 3D Spatial Images

Researchers have used the Kalman filter to extract 3D spatial information from multisource remote sensing optical stereo image pairs.

By jointly considering the extraction of position information, recovery of spatial structure, and topological constraints, the Kalman filter improves the accuracy and reliability of 3D spatial data.

### Photon-Counting Integral Imaging

In extremely low-light conditions, the Kalman filter is applied to photon-counting integral imaging.

It estimates distance and human motion by predicting future values based on observations and gain values.

By combining measurements and predictions, it enhances the quality of 3D visualizations under challenging lighting conditions.

In summary, the Kalman filter plays a crucial role in 3D image capturing by handling noisy data, predicting system states, and improving the accuracy of reconstructed 3D scenes. Its ability to combine predictions and measurements makes it valuable for various computer vision tasks.

Useful for dynamic systems and time-series data, the Kalman filter is a recursive solution to the least-squares problem that updates estimates as new data arrives. It is represented as:

$${\hat{x}}_{k} {\vert}_{k} = \hat{χ} k \vert {k}_{- 1} + {K}_{k} ( yk - {H}_{k} {\hat{x}}_{k} {\vert}_{k - 1} )$$

wherein:
\hat{χ}k|k₋₁ is the estimate of the state at time k given knowledge of the process prior to time k,
yk is the measurement at time k,
Kₖ is the Kalman gain,
Hₖ is the design matrix.

### 3. Total Variation Denoising

Especially useful in image processing for noise reduction without losing edges by minimizing the total variation of the image:

$${min}_{u} \{ {\int }_{Ω} \vert Du \vert + \frac{λ}{2} {\int }_{Ω} {( u - f )}^{2} \}$$

wherein:
Ω is the domain of the image,
Du represents the gradient of the image,
λ is the regularization parameter,
f is the original noisy image.

### 4. RANSAC (Random Sample Consensus)

A method for robust fitting of models in the presence of many data outliers. In geometric reconstruction, it helps to ignore points that do not fit well and could be considered noise:

Model parameters-RANSAC (Data points, Model, Threshold) Model parameters=RANSA C (Data points,Model, Threshold)

### 5. Wiener Filter

An optimal filter for signal processing in the presence of additive noise and blurring:

$$G ( u , v ) = \frac{{H}^{*} ( u , v ) S ( u , v )}{{\vert H ( u , v ) \vert}^{2} + \frac{{S}_{n} ( u , v )}{S ( u , v )} )} F ( u , v )$$

wherein:
H(u,v) is the degradation function,
S(u,v) and Sₙ(u,v) are the power spectrum of the signal and noise respectively,
F(u,v) is the observed image with noise,
G(u,v) is the estimated noise-free image.

### 6. Application to Noise Correction in Geometric Reconstruction

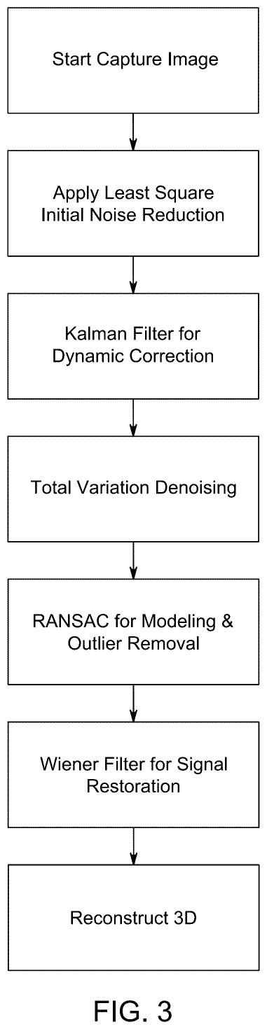

These equations and methods can be applied to adjust and correct the raw data obtained from the imaging system, accounting for random noise and systematic errors (see FIG. 3). FIG. 3 provides a flowchart to utilize for adjustments based on noise and errors. The goal is to refine the 3D reconstruction of a patient's torso, or in this case, specifically addressing noise that might affect the measurement of the patient's nose or other critical anatomical features. The choice of method may depend on the type of noise and the specific requirements of the system in terms of speed and accuracy.

### Robust AI Application

When scanning a patient with a structured light system in different clinical scenarios, an AI system must be capable of distinguishing between relevant anatomical features and extraneous objects or conditions in the scene, such as blankets or oxygen masks. This task involves a combination of image preprocessing, feature extraction, and machine learning algorithms that can adaptively learn to recognize and filter out irrelevant variables. Provided herewith are various techniques and associated mathematics that an AI system can use:

### 1. Image Preprocessing

To enhance the features of interest and reduce noise or irrelevant details, image preprocessing techniques such as filtering and thresholding are applied.

Gaussian Blur: To smooth out irrelevant details, Gaussian blur can be used.

$$G ( x , y ) = \frac{1}{2 {πσ}^{2}} {e}^{- \frac{{x}^{2} + {y}^{2}}{2 {σ}^{2}}}$$

where G is the Gaussian kernel, σ is the standard deviation of the distribution, and x, y are the distances from the origin in the horizontal and vertical axes, respectively.

Thresholding: It converts grayscale images to binary (black and white) images, keeping only the intensities above a certain threshold.

$$\{ \begin{matrix}1 & if I ( x , y ) \geq threshold \\ 0 & otherwise\end{matrix}$$

where I is the image intensity.

### 2. Feature Extraction

This step involves identifying and isolating important characteristics or features within the images that are crucial for the analysis.

Edge Detection (Sobel Operator): To find edges which can represent boundaries of blankets or masks or other variables.

For the horizontal gradient Gₓ:

$${G}_{x} = [ \begin{matrix}- 1 & 0 & 1 \\ - 2 & 0 & 2 \\ - 1 & 0 & 1\end{matrix} ] * A$$

For the vertical gradient G_{y}

$${G}_{y} = [ \begin{matrix}- 1 & - 2 & - 1 \\ 0 & 0 & 0 \\ 1 & 2 & 1\end{matrix} ] * A$$

The “*” represents the convolution operation between each kernel and the image A. The result of these convolutions are two matrices Gₓ and G_{y} that contain the gradients of the image A in the horizontal and vertical directions, respectively. These gradients can then be combined to find the magnitude of the gradient at each point in the image, which corresponds to the strength of the edges at those points:

$$G = \sqrt{{G}_{x}^{2} + {G}_{y}^{2}}$$

This equation gives us the edge magnitude, which is used to detect the presence and location of edges within the image.

### 3. Machine Learning for Object Recognition

AI uses machine learning algorithms to differentiate between the patient's body and other objects.

Convolutional Neural Networks (CNNs): Specialized in image recognition, CNNs can be trained to recognize and differentiate the patient's body from other objects.

The operation of a convolutional layer in a CNN can be mathematically expressed as:

$$( f * k ) ( i , j ) = {\sum }_{m} {\sum }_{n} f ( m , n ) k ( i - m , j - n )$$

where f is the image, and k is the kernel or filter applied to the image.

Segmentation Models (U-Net): Used for separating different parts of the image into relevant categories or segments. The U-Net architecture excels in medical image segmentation.

### 4. Classification and Regression Trees (CART)

CART algorithms can classify pixels or regions of the image based on features derived from the preprocessing and feature extraction steps.

$${G}_{ini} ( D ) = 1 - {\sum }^{m} i = {1}^{{p}^{2}} i$$

where D is a dataset, pi is the probability of an item belonging to class i, and m is the number of classes.

### Application in Scene Understanding

The AI system can use these techniques in tandem to analyze the scene. For instance, if a patient is under a blanket, edge detection can help outline the blanket, and the CNN can recognize it as a non-anatomical object. CART algorithms can further classify regions identified by the CNN to ensure accurate segmentation. The blanket and oxygen mask, once identified, can be excluded from the volumetric analysis performed by the structured light system, thus maintaining focus on relevant anatomical data.

By combining these techniques, an AI system can create robust models capable of adapting to varying clinical scenarios, ensuring that the measurements captured for mechanical ventilation settings are accurate and reliable.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure. Various changes and advantages may be made in the above disclosure without departing from the spirit and scope thereof.

1. A method for determining personalized ventilation settings for a patient in need of ventilation support, comprising the steps of:
using an emitting structured light to capture 3D spatial data of a patient's torso;
analyzing the data with a computational analysis program to compute critical measurements including torso height, girth, and volume; and
utilizing these measurements to recommend personalized mechanical ventilation settings.

2. The method of claim 1, wherein the device comprises a hand-held device.

3. The method of claim 1, wherein the structured light is in the near-infrared spectrum, approximately around 940 nm, to maximize penetration and minimize interference from ambient light.

4. The method of claim 1, wherein the computational analysis program processes the data to calculate personalized settings, specifically tailoring ventilation parameters based on the unique anatomical data of the patient.

5. The method of claim 1, wherein the patient is a woman.

6. The method of claim 1, wherein the patient is an infant.

7. The method of claim 1, wherein the patient is an adolescent.

8. A system for use in practicing the method of claim 1, comprising a device configured to emit structured light to capture 3D special data of a patient's torso and other anatomical features; and
a computer configured to store and analyze the data utilizing a program to algorithmically compute critical measurements including torso height, girth and volume.

9. The system of claim 8, wherein the device is a hand-held device.

10. The system of claim 8, wherein the structured light is near-infrared (IR) technology.

11. The system of claim 10, wherein the structured light is in the near-infrared spectrum of approximately 940 nm.

12. The system of claim 8, wherein the computational analysis program processes the data within an output device utilizing a processor to calculate personalized settings and store said computations in non-transitory memory, said computations specifically tailoring ventilation parameters based on the unique anatomical data of the patient.

13. The system of claim 8, wherein the patient is a woman.

14. The system of claim 8, wherein the patient is an infant or an adolescent.

15. A handheld device for measuring a patient's attributes prior to intubation, wherein the device obtains measurements and comprises a processor and non-transitory memory.

16. The device of claim 15, wherein structured light near-infrared (IR) technology scans and creates 3D maps of a patient's body.

17. The device of claim 15, wherein the device provided data can be utilized to calculate ventilation settings based on the patient's physiology.

18. The device of claim 15, wherein data can be shared with other output devices.

19. The device of claim 18, wherein the output devices may be selected from a tablet, a smartphone or a computer.

20. The device of claim 15, wherein the measurable attributes are torso girth, height and volume.
